(19) United States
(12) Patent Application Publication
(10) Pub. No.: US 2024/0000378 A1
(43) Pub. Date: Jan. 4, 2024

(54) SYSTEM AND METHOD FOR QUANTIFYING AN INJURY RECOVERY STATE

(71) Applicant: Orpyx Medical Technologies Inc., Calgary (CA)

(72) Inventors: SAMUEL CARL WILLIAM BLADES, VICTORIA (CA); HUNTER BROOKE MARRIOTT, OTTAWA (CA)

(21) Appl. No.: 18/335,493

(22) Filed: Jun. 15, 2023

Related U.S. Application Data
(60) Provisional application No. 63/356,951, filed on Jun. 29, 2022.

(51) Int. Cl. A61B 5/00; A61B 5/103

(52) U.S. Cl. CPC A61B 5/4842; A61B 5/1038; A61B 5/6807; A61B 5/7246; A61B 2503/10; A61B 2505/09; A61B 2562/0219

(57) ABSTRACT

A system, method and computer program product for quantitatively assessing an injury recovery state for a user. Sensor data can be obtained from a plurality of sensors positioned to contact the user while the user performs a set of specified activities. The sensors can be mounted to a user using one or more wearable devices. Metric values can be calculated from the sensor data obtained while the user performs the specified activities. The metric values can be compared to baseline values for the user. A quantified injury recovery state value can be calculated based on the comparison of the metric values with the respective baseline values. The quantified injury recovery state value can provide a quantitative assessment of a user's injury recovery or injury risk that can be used to provide activity recommendations and/or return-to-play (RTP) decisions in athletics.

### CROSS-REFERENCE TO RELATED APPLICATIONS

This application claims the benefit of priority of U.S. Provisional Application No. 63/356,951 filed Jun. 29, 2022, which is incorporated herein by reference.

### FIELD

This document relates to systems and methods for processing data from sensors monitoring human movement or human activity. In particular, this document relates to quantifying an injury recovery state for a user using sensor data from a wearable device worn by the user.

### BACKGROUND

US Patent Publication No. 2020/0108291 (Piazza et al.) provides a system, method, and computer-readable media to collect and analyze the physical performance of an individual and structure and generate an athletic protocol. Athletic assessment devices perform an athletic assessment of an individual, including kinetic, neurological, musculoskeletal and aerobic capacity. Athletic data is collected and stored in a digital data storage medium of a computer system having a display, and a central processing unit (CPU) operable with programming executes one or more analytical algorithms to assess injury risk levels of said individual based on said athletic data. Data mining algorithms using artificial intelligence are used to execute predictive modeling and analytics. The resultant athletic protocol includes information to correct imbalances to treat and/or prevent injuries, corrective exercises, performance training exercises, and protocols to maximize physical performance.

U.S. Pat. No. 11,232,860 (McNair) provides systems and methods for injury characterization including detecting matches of an individual subject's record (such as an athlete's record) with collections of other subjects' records, based on serial, longitudinal patterns, for facilitating athlete health and training, preventive and rehabilitation medicine, and risk management in athletics. In an embodiment, time series are formed from information pertaining to successive longitudinal episodes of injury and the circumstances in which the injuries were incurred; calculating time-series K-nearest-neighbor clusters and distances for each such combination; determining the cluster to which a given candidate player injury record is nearest or belongs, and prescribing an injury-risk reduction intervention specific to the plurality of hazards that are characteristic of trajectories that are members of that cluster and that are deemed to be relevant to reducing or mitigating those hazards and thereby are efficacious in preventing subsequent injuries that are prevalent in that trajectory cluster.

### SUMMARY

The following summary is intended to introduce the reader to various aspects of the detailed description, but not to define or delimit any invention.

A system, method and computer program product for quantitatively assessing an injury recovery state for a user. Sensor data can be obtained from a plurality of sensors positioned to contact the user while the user performs a set of specified activities. The sensors can be mounted to a user using one or more wearable devices. Metric values can be calculated from the sensor data obtained while the user performs the specified activities. The metric values can be compared to baseline values for the user. A quantified injury recovery state value can be calculated based on the comparison of the metric values with the respective baseline values. The quantified injury recovery state value can provide a quantitative assessment of a user's injury recovery or injury risk that can be used to provide activity recommendations and/or return-to-play (RTP) decisions in athletics.

According to some aspects, a method for quantifying an injury recovery state for a user includes obtaining a plurality of sensor readings from a corresponding plurality of sensors provided in a wearable device, and while the wearable device is worn by the user while performing one or more specified activities; identifying the one or more specified activities; for each specified activity of the one or more specified activities, calculating at least one metric value for the user based on activity-specific sensor data corresponding to that specified activity from the plurality of sensor readings, wherein each metric value corresponds to a particular metric of interest associated with that specified activity; comparing each metric value to a corresponding baseline metric value for the user; and calculating a quantified injury recovery state value based on the comparison of each metric value and the corresponding baseline metric value.

The quantified injury recovery state value can be output.

The plurality of sensor readings can be obtained after the user has incurred an injury.

Each baseline metric value can be calculated using baseline sensor readings obtained prior to the user incurring the injury.

The injury may be to a first lower limb of the user; each metric value can be calculated using first limb sensor readings in the plurality of sensor readings, the first limb sensor readings received from sensors positioned to monitor the first lower limb; and each baseline metric value can be calculated using second limb sensor readings in the plurality of sensor readings, the second limb sensor readings received from sensors positioned to monitor a second lower limb of the user, where the second lower limb is uninjured.

The quantified injury recovery state value can be a current injury recovery state value determined using at least one baseline deviation value, each baseline deviation value calculated from the comparison of a given metric value and the corresponding baseline metric value.

Each baseline deviation value can be calculated as a percentage deviation.

The at least one baseline deviation value can include a plurality of baseline deviation values and the current injury recovery state value can be determined using a weighted sum of the plurality of baseline deviation values.

The method can include, for each specified activity, determining at least one previous metric value for the user, each previous metric value calculated using previous sensor readings previously obtained from the user while performing the one or more specified activities; comparing each previous metric value to the corresponding baseline metric value for the user; and determining a previous injury recovery state value using at least one previous baseline deviation value, each previous baseline deviation value calculated from the comparison of a given previous metric value and the corresponding baseline metric value.

The method can include determining an injury improvement value by comparing the current injury recovery state value and the previous injury recovery state value.

The method can include determining an injury improvement rate using the current injury recovery state value and the previous injury recovery state value.

The method can include identifying a potential recovery state value for the user using the at least one baseline metric value; and calculating a predicted recovery time using the current injury recovery state value and the previous injury recovery state value.

Each specified activity can be identified using the sensor data.

Each specified activity can be identified by inputting the activity-specific sensor data corresponding to that specified activity to an activity classification model.

Each specified activity can be identified from a predefined list of potential activities.

The method can include identifying the activity-specific sensor data corresponding to each specific activity in the sensor readings; for each specific activity, determining whether the specified activity was performed correctly or incorrectly based on the activity-specific sensor data; and only calculating the at least one metric value in response to determining that the specified activity was performed correctly.

Each specified activity can be identified from a predefined list of potential activities and the method can include, for each specified activity: evaluating the sensor readings to identify activity-specific sensor data corresponding to one of the potential activities; identifying the one of the potential activities as that specified activity; and determining that the specified activity was performed correctly in response to determining that the activity-specific sensor data corresponds to the one of the potential activities.

The plurality of sensor readings can include a first set of sensor readings corresponding to the user performing the one or more specified activities in a non-fatigued state and a second set of sensor readings corresponding to the user performing the one or more specified activities in a fatigued state, and the method can include, for each specified activity: calculating at least one non-fatigued metric value for the user based on activity-specific sensor data corresponding to that specified activity from the first set of sensor readings; calculating at least one fatigued metric value for the user based on activity-specific sensor data corresponding to that specified activity from the second set of sensor readings; and calculating the quantified injury recovery state value using the at least one fatigued metric value.

The plurality of sensors can include a force-sensing element.

The plurality of sensors can include an inertial measurement unit.

The at least one metric value can include at least one of: a center of pressure path value, a center of pressure velocity value, a rate of force development value, a peak force value, a jump height value, an impulse value, a rate of force dissipation value, a time to stabilization value, a distance value, or a ground contact time vs flight time value.

The wearable device can be footwear.

The footwear can be an insole.

The method can include comparing at least one of the metric values to a corresponding metric value threshold; and outputting a value threshold satisfaction output indicating whether the at least one of the metric values satisfies the corresponding metric value threshold.

According to some aspects, a system for quantifying an injury recovery state for a user includes: a plurality of sensors mountable to the user, wherein the plurality of sensors is configured to obtain a plurality of sensor readings from the user while the user is performing one or more specified activities; a memory configured to store the plurality of sensor readings; and one or more processors configured to: identify the one or more specified activities; for each specified activity of the one or more specified activities, calculate at least one metric value for the user based on activity-specific sensor data corresponding to that specified activity from the plurality of sensor readings, wherein each metric value corresponds to a particular metric of interest associated with that specified activity; compare each metric value to a corresponding baseline metric value for the user; and calculate a quantified injury recovery state value based on the comparison of each metric value and the corresponding baseline metric value.

The one or more processors can be configured to output the quantified injury recovery state value.

The plurality of sensor readings can be obtained after the user has incurred an injury.

The one or more processors can be configured to calculate each baseline metric value using baseline sensor readings obtained prior to the user incurring the injury.

The injury may be to a first lower limb of the user and the one or more processors can be configured to: calculate each metric value using first limb sensor readings in the plurality of sensor readings, the first limb sensor readings received from sensors positioned to monitor the first lower limb; and calculate each baseline metric value using second limb sensor readings in the plurality of sensor readings, the second limb sensor readings received from sensors positioned to monitor a second lower limb of the user, wherein the second lower limb is uninjured.

The one or more processors can be configured to determine the quantified injury recovery state value as a current injury recovery state value using at least one baseline deviation value, where each baseline deviation value is calculated from the comparison of a given metric value and the corresponding baseline metric value.

The one or more processors can be configured to calculate each baseline deviation value as a percentage deviation.

The at least one baseline deviation value can include a plurality of baseline deviation values and the one or more processors can be configured to determine the current injury recovery state value using a weighted sum of the plurality of baseline deviation values.

The one or more processors can be configured to: for each specified activity, determine at least one previous metric value for the user, each previous metric value calculated using previous sensor readings previously obtained from the user while performing the one or more specified activities; compare each previous metric value to the corresponding baseline metric value for the user; and determine a previous injury recovery state value using at least one previous baseline deviation value, each previous baseline deviation value calculated from the comparison of a given previous metric value and the corresponding baseline metric value.

The one or more processors can be configured to determine an injury improvement value by comparing the current injury recovery state value and the previous injury recovery state value.

The one or more processors can be configured to determine an injury improvement rate using the current injury recovery state value and the previous injury recovery state value.

The one or more processors can be configured to: identify a potential recovery state value for the user using the at least one baseline metric value; and calculate a predicted recovery time using the current injury recovery state value and the previous injury recovery state value.

The one or more processors can be configured to identify each specified activity using the sensor data.

The one or more processors can be configured to identify each specified activity by inputting the activity-specific sensor data corresponding to that specified activity to an activity classification model.

The one or more processors can be configured to identify each specified activity from a predefined list of potential activities.

The one or more processors can be configured to: identify the activity-specific sensor data corresponding to each specific activity in the sensor readings; for each specific activity, determine whether the specified activity was performed correctly or incorrectly based on the activity-specific sensor data; and only calculate the at least one metric value in response to determining that the specified activity was performed correctly.

The one or more processors can be configured to: identify each specified activity from a predefined list of potential activities and, for each specified activity, evaluate the sensor readings to identify activity-specific sensor data corresponding to one of the potential activities; identify the one of the potential activities as that specified activity; and determine that the specified activity was performed correctly in response to determining that the activity-specific sensor data corresponds to the one of the potential activities.

The plurality of sensor readings can include a first set of sensor readings corresponding to the user performing the one or more specified activities in a non-fatigued state and a second set of sensor readings corresponding to the user performing the one or more specified activities in a fatigued state, and the one or more processors can be configured to, for each specified activity: calculate at least one non-fatigued metric value for the user based on activity-specific sensor data corresponding to that specified activity from the first set of sensor readings; calculate at least one fatigued metric value for the user based on activity-specific sensor data corresponding to that specified activity from the second set of sensor readings; and calculate the quantified injury recovery state value using the at least one fatigued metric value.

The plurality of sensors can include a force-sensing element.

The plurality of sensors can include an inertial measurement unit.

The at least one metric value can include at least one of: a center of pressure path value, a center of pressure velocity value, a rate of force development value, a peak force value, a jump height value, an impulse value, a rate of force dissipation value, a time to stabilization value, a distance value, or a ground contact time vs flight time value.

The wearable device can be footwear.

The footwear can be an insole.

The one or more processors can be configured to compare at least one of the metric values to a corresponding metric value threshold; and output a value threshold satisfaction output indicating whether the at least one of the metric values satisfies the corresponding metric value threshold.

According to some aspects, there is provided a non-transitory computer readable medium storing computer-executable instructions, which, when executed by a computer processor, cause the computer processor to carry out a method for quantifying an injury recovery state for a user. The method includes obtaining a plurality of sensor readings from a corresponding plurality of sensors provided in a wearable device, and while the wearable device is worn by the user while performing one or more specified activities; identifying the one or more specified activities; for each specified activity of the one or more specified activities, calculating at least one metric value for the user based on activity-specific sensor data corresponding to that specified activity from the plurality of sensor readings, wherein each metric value corresponds to a particular metric of interest associated with that specified activity; comparing each metric value to a corresponding baseline metric value for the user; and calculating a quantified injury recovery state value based on the comparison of each metric value and the corresponding baseline metric value.

The non-transitory computer readable medium can store computer-executable instructions, which, when executed by a computer processor, cause the computer processor to carry out the method for quantifying an injury recovery state for a user, where the method is described herein.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a block diagram illustrating an example of a system for quantifying an injury recovery state for a user;

FIG. 2 is a diagram illustrating an example of a wearable device incorporating a sensing unit that can be used in the system of FIG. 1;

FIG. 3 is a flowchart illustrating an example of a method for quantifying an injury recovery state for a user;

FIG. 4 is a plot showing an example of baseline deviation injury recovery state values over time as a user recovers from an injury;

FIG. 5 is a plot showing an example of an injury improvement value between points in time as a user recovers from an injury;

FIG. 6 is a plot showing an example of an injury improvement rate curve over time as a user recovers from an injury; and

FIG. 7 is a plot showing an example of a predicted recovery time for a user recovering from an injury.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending on the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Terms of degree such as “substantially”, “about”, and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The systems, methods, and devices described herein may be implemented as a combination of hardware or software. In some cases, the systems, methods, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming. Accordingly, the program code may be written in any suitable programming language such as Python or C, for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The present disclosure relates in general to systems, methods, and computer program products that can be used to quantitatively assess an injury recovery state for an individual. The systems, methods and computer program products can evaluate the biomechanics of a user's lower limbs while the user performs specified exercise activities. An injury recovery state can be quantified for the individual based on this evaluation. The quantified injury recovery state value can then be used to inform decisions relating to further activities and exercises that may be recommended to or performed by the individual.

Clinicians typically use subjective tools such as questionnaires and joint laxity tests to assess if an individual is ready to return to play a given activity (referred to as RTP decisions). Where an individual is participating in a competitive activity or is a member of a competitive team, clinicians may feel pressured by team sponsors, coaches, and others to arrive at certain RTP decisions which may not be in the best interest of the individual's health. The present disclosure provides a quantitative way of assessing an individual's injury recovery state that can be used to guide further exercise by the user and/or to further inform clinicians about an RTP decision.

The systems, methods and computer program products described herein can evaluate biomechanical data relating to the performance of one or more specified activities by a user. The biomechanical data can be determined based on sensor readings acquired in relation to the user performing the one or more specified activities. The systems, methods and computer program products described herein can be used to quantify an injury recovery state for the user based on metric values determined for the user's performance of the specified activities.

Metric values can be calculated from sensor data obtained while the user is performing a specified activity. Metric values may be determined for a variety of different specified activities. Examples of specified activities can include exercises such as walking, running, squatting, lunging, jumping and so forth.

The sensor readings can be obtained from a plurality of sensors positioned in a wearable device worn by the user while the user performs an activity. The sensors can be attached to, or contained within, one or more wearable devices to measure and monitor data relating to movement or activity of an individual. The measured data from the sensors can be used to determine metric values relating to the performance of the activity by the user. The metric values can be compared to baseline values for the same metrics in order to calculate a quantified injury recovery state value for the user.

The sensors can include a plurality of force sensors positioned underfoot while the user is performing the specified activity. The force sensors can be provided in a wearable device in the form of an insole of a shoe, a sock, or within the footwear worn by the individual. The force data acquired by the force sensors can be used to determine the level of force applied by an individual's foot when performing various activities such as walking, running, or jumping for example. This force data can be used to derive additional force derivatives or force-based metrics, such as the force output, mean force or the peak force applied by the foot. The force data, and other data derived therefrom, can be used to determine metric values relating to the user's performance in the specified activities.

The force sensor data can be used to determine force values for the user while performing one or more specified activities. Directly measuring the force (or pressure) applied by an individual using underfoot force sensors (as opposed to deriving the force data from other sensors such as accelerometers) can contribute to more accurate calculations of force-derived metric values. Directly measuring the force applied by an individual using underfoot force sensors also allows force to be measured during static conditions (e.g. while a user is standing or performing a lunge). As used herein, the term “force” is used broadly and can refer to raw force (i.e. with units of N), or pressure resulting from a raw force (i.e. with units of N/m 2).

Metric values can be calculated from the sensor readings obtained while the user is performing a specified activity. The metric values can be compared to baseline metric values that are determined for the user based on baseline sensor data obtained from the same user. Differences (e.g. percent differences) between the metric values and the baseline values can be used to calculate a quantified injury recovery state value for the user. The quantified injury recovery state value can be used to make decisions or recommendations relating to further activities for the user.

Various different types of metric values can be calculated from the sensor readings. Examples of metric values that can be determined based on the sensor readings obtained while the user is performing a specified activity include a center of pressure (COP) path displacement value, a center of pressure velocity value (e.g. an anterior-posterior (AP) COP velocity value and/or a medial lateral (ML) COP velocity value), a rate of force development value, a peak force value, a jump height value, an impulse value, a rate of force dissipation value, a time to stabilization value, a distance value, or a ground contact time vs flight time value.

The systems, methods and devices described herein can also include one or more inertial measurement units (IMUs). Each IMU may be associated with a corresponding plurality of force sensors. That is, each IMU can be configured to collect inertial measurement data relating to movement of the same foot under which the force sensors are positioned. IMU data from the one or more IMUs can be used to determine metric values for the user.

The systems, methods and devices described herein can also be used to acquire sensor data for both of an individual's feet at the same time. In some cases, this may require a separate plurality of force sensors for each foot (e.g. where the force sensors are incorporated into a wearable device).

Where an IMU is included in the system and method, a separate IMU can be provided for each foot. This allows the IMU to collect inertial measurement data relating to movement of that foot. The inertial measurement data specific to the foot can then be used to calculate metric values for that foot.

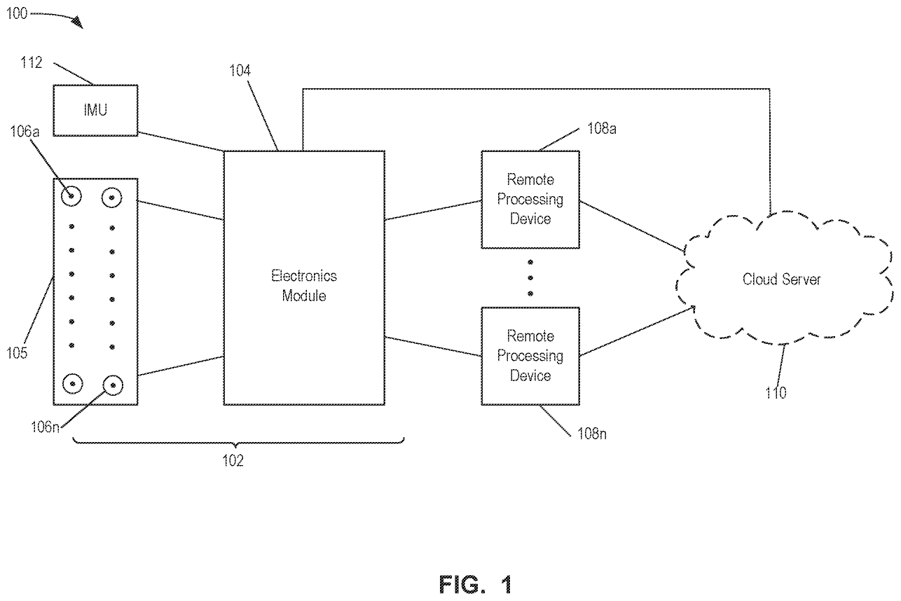

Referring now to FIG. 1, shown therein is a block diagram illustrating an example system 100 that can be used to quantify an injury recovery state for a user. System 100 includes a plurality of sensors positionable underfoot of an individual performing an activity or other type of movement. The sensors may be provided using one or more wearable devices that can be worn while the user is performing one or more exercise activities.

System 100 includes an input unit 102 (also referred to herein as an input device), one or more processing devices 108 (also referred to herein as a receiving device or an output device) and an optional remote cloud server 110. As will be described in further detail below, the input unit 102 may for example be combined with, or integrated into, a carrier unit such as a wearable device or a piece of fitness equipment.

Input unit 102 generally includes a sensing unit 105. The sensing unit 105 can include a plurality of sensors 106a-106n. The plurality of sensors 106a-106n can be configured to collect force sensor data from underneath the foot of an individual.

In the example shown, input unit 102 includes an inertial measurement unit (IMU) 112. IMU 112 can include one or more sensors for measuring the position and/or motion of the wearable device. For example, IMU 112 may include sensors such as one or more of a gyroscope, accelerometer (e.g., a three-axis accelerometer), magnetometer, orientation sensor (for measuring orientation and/or changes in orientation), angular velocity sensor, and inclination sensor.

The IMU 112 can also be positioned underneath a foot of an individual. However, the IMU 112 need not be positioned underfoot so long as the IMU 112 can collect inertial measurement data relating to the position and/or motion of the individual's lower limb (e.g. foot, ankle, knee, shin, and/or thigh).

The carrier unit can be configured to position the sensors 106 in contact with (or in close proximity to) an individual's body to allow the sensors 106 to measure an aspect of the activity being performed by the individual. The plurality of sensors 106a-106n may be configured to measure a particular sensed variable at a location of an individual's body when the carrier unit is engaged with the individual's body (e.g. when the individual is wearing a wearable device containing the sensors 106 or when the individual is using fitness equipment containing the sensors 106). In system 100, the plurality of sensors 106a-106n can be arranged to measure force underneath the foot (underfoot) of an individual. As noted above, the IMU 112 can be arranged to measure data relating to the position and/or motion of the individual's lower limb.

Optionally, the system 100 can include one or more IMUs 112 arranged to measure data relating to the position and/or motion of other parts of the user's body, such as the user's center of mass (e.g. proximate the user's sacrum), the user's waist, the user's tibia, the user's knee etc. This may be particularly useful in assessing certain specific activities. For example, an IMU positioned at a user's waist may acquire IMU data usable to accurately measure a jump height.

In some examples, the carrier unit may include one or more wearable devices. The wearable devices can be manufactured of various materials such as fabric, cloth, polymer, or foam materials suitable for being worn close to, or in contact with, a user's skin. All or a portion of the wearable device may be made of breathable materials to increase comfort while a user is performing an activity.

In some examples, the wearable device may be formed into a garment or form of apparel such as a sock, a shoe, an insole, or a sleeve. Some wearable devices such as socks may be in direct contact with a user's skin. Some wearable devices, such as shoes, may not be in direct contact with a user's skin but still positioned within sufficient proximity to a user's body to allow the sensors to acquire the desired readings.

In some cases, the wearable device may be a compression-fit garment. The compression-fit garment may be manufactured from a material that is compressive. A compression-fit garment may minimize the impact from “motion artifacts” by reducing the relative movement of the wearable device with respect to a target location on the individual's body. In some cases, the wearable device may also include anti-slip components on the skin-facing surface. For example, a silicone grip may be provided on the skin-facing surface of the wearable device to further reduce the potential for motion artifacts.

The wearable device can be worn on a foot. For example, the wearable device may be a shoe, a sock, or an insole, or a portion of a shoe, a sock, or an insole. The wearable device may include a deformable material, such as foam. This may be particularly useful where the wearable device is a shoe or insole.

The plurality of sensors 106a-106n can be positioned to acquire sensor readings from specified locations on an individual's body (via the arrangement of the sensors on the carrier unit). The sensors 106 can be integrated into the material of the carrier unit (e.g. integrated into a wearable device or fitness equipment). Alternatively, the sensors 106 can be affixed or attached to the carrier unit, e.g. printed, glued, laminated or ironed onto a surface, or between layers, of a wearable device or fitness equipment.

For clarity, the below description relates to a carrier unit in the form of an insole. The insole carrier unit may be provided in various forms, such as an insert for footwear, or integrated into a shoe. However, other carrier units may be implemented using the systems and methods described herein, such as the wearable devices described above.

Incorporating the sensing unit 105 (and optionally the IMU 112) into a carrier unit in the form of a wearable device may be desirable as it allows the user's performance in various activities to be assessed at various locations and without requiring specifically configured fitness equipment. This allows a user's performance to be evaluated while performing specified activities that generally occur outside of a laboratory environment, such as specific sporting activities (e.g. activities typically associated with soccer, football, hockey, cross-country running etc.), occupational activities, or more general daily living activities for example. Accordingly, the user's performance can be evaluated based on the same conditions in which the individual would normally perform the specified activity. The collection of the biomechanical data in a “field” setting (i.e. outside of a specified laboratory setting) allows a user to perform the specified activity as they ordinarily would, without needing specific foot scan/gait analysis tests. This may allow an individual's injury recovery state to be quantified for any lower limb injury, regardless of how the injury is sustained, including impact and non-impact injuries.

Integrating the plurality of sensors into a wearable device also allows biomechanical data to be collected while a user is participating in an event such as training, practice or a competition. This may allow the user's injury recovery state to be quantified in real-time to identify the potential for re-injury and/or fatigue-related injury for example.

In addition, the biomechanical data can be used to identify changes in the metric values while a user is performing an activity. The changes can be identified to the user as an indication that their technique has changed or that they are becoming fatigued and may have an increased risk of injury or re-injury. The changes can also be identified to the user as an indication that they are recovering from an injury or that they are applying a compensatory technique to account for the injury.

The below description relates to an insole in which the plurality of sensors 106 are force sensors. Various types of force sensors may be used, such as force sensing resistors (also referred to as sensing elements), pressure sensors, piezoelectric tactile sensors, elasto-resistive sensors, capacitive sensors or more generally any type of force sensor that can be integrated into a wearable device or fitness equipment capable of collecting force data underfoot.

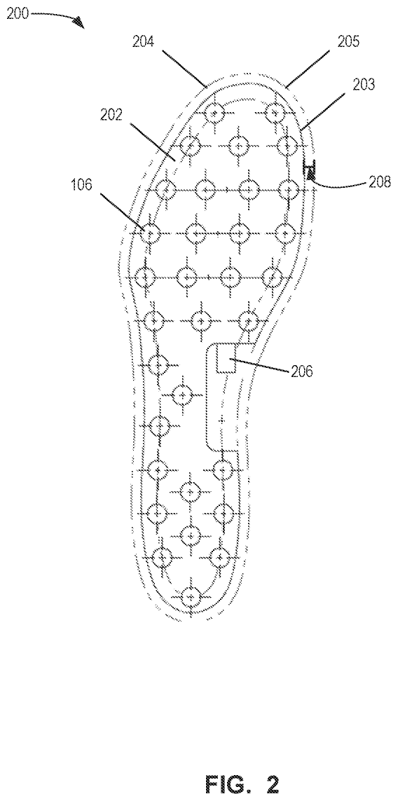

The plurality of sensors 106 may be arranged into a sensor array. As used herein, the term sensor array refers to a series of sensors arranged in a defined grid. The plurality of sensors 106 can be arranged in various types of sensor arrays. For example, the plurality of sensors 106 can be provided as a set of discrete sensors (see e.g. FIG. 2). A discrete sensor is an individual sensor that acquires a sensor reading at a single location. A set of discrete sensors generally refers to multiple discrete sensors that are arranged in a spaced apart relationship in a sensing unit.

Sensors 106a-106n may be arranged in a sparse array of discrete sensors that includes void locations where no sensors 106 are located. Alternatively, sensors 106a-106n may be arranged in a continuous or dense sensor array in which sensors 106 are arranged in a continuous, or substantially continuous manner, across the grid.

Discrete sensors can provide an inexpensive alternative to dense sensor arrays for many applications. However, because no sensors are positioned in the interstitial locations between the discrete sensors and the void locations external to the set of discrete sensors, no actual sensors readings can be acquired for these locations. Accordingly, depending on the desired resolution for the force sensor data, sensor readings may be estimated (rather than measured) at the interstitial locations and at the void locations external to the set of discrete sensors in order to provide sensor data with similar resolution to a dense sensor array. Alternatively, where lower resolution force sensor data is sufficient, sensor readings may not necessarily be estimated.

Various interpolation and extrapolation techniques may be used to estimate sensor values at interstitial locations and external void locations. In some cases, sensor values may be estimated using the methods for synthesizing sensor data described in Applicant's co-pending patent application Ser. No. 17/988,468 filed on Nov. 16, 2022 entitled “SYSTEM AND METHOD FOR SYNTHESIZING SENSOR READINGS”, the entirety of which is incorporated herein by reference. In some cases, sensor values may be estimated using the methods for synthesizing sensor data described in Applicant's co-pending patent application Ser. No. 18/183,642 filed on Mar. 14, 2023 entitled “SYSTEM AND METHOD FOR DETERMINING USER-SPECIFIC ESTIMATION WEIGHTS FOR SYNTHESIZING SENSOR READINGS”, the entirety of which is incorporated herein by reference.

System 100 can be configured to implement methods of quantifying an injury recovery state for an individual. The methods of quantifying an injury recovery state can be implemented using a controller of the input device 102, a remote processing device 108, or cloud server 110.

As shown in FIG. 1, input unit 102 includes an electronics module 104 coupled to the plurality of sensors 106 and to IMU 112. In some cases, the electronics module 104 can include a power supply, a controller, a memory, a signal acquisition unit operatively coupled to the controller and to the plurality of sensors 106 (and to IMU 112), and a wireless communication module operatively coupled to the controller.

Generally, the sensing unit refers to the plurality of sensors 106 and the signal acquisition unit. The signal acquisition unit may provide initial analog processing of signals acquired using the sensors 106, such as amplification. The signal acquisition unit may also include an analog-to-digital converter to convert the acquired signals from the continuous time domain to a discrete time domain. The analog-to-digital converter may then provide the digitized data to the controller for further analysis or for communication to a remote processing device 108 or remote cloud server 110 for further analysis.

Optionally, the electronics module 104 may include a controller or other processing device configured to perform the signal processing and analysis. In such cases, the controller on the electronics module may be configured to process the received sensor readings in order to determine synthesized sensor readings and/or calculate metric values. In some cases, the controller may be coupled to the communication module (and thereby the sensing unit) using a wired connection such as Universal Serial Bus (USB) or other port.

The electronics module 104 can be communicatively coupled to one or more remote processing devices 108a-108n, e.g. using a wireless communication module (e.g., Bluetooth, Bluetooth Low-Energy, WiFi, ANT+ IEEE 802.11, etc.). The remote processing devices 108 can be any type of processing device such as (but not limited to) a personal computer, a tablet, and a mobile device such as a smartphone, a smartwatch or a wristband. The electronics modules 104 can also be communicatively coupled to remote cloud server 110 over, for example, a wide area network such as the Internet.

Each remote processing device 108 and optional remote cloud server 110 typically includes a processing unit, an output device (such as a display, speaker, and/or tactile feedback device), a user interface, an interface unit for communicating with other devices, Input/Output (I/O) hardware, a wireless unit (e.g. a radio that communicates using CDMA, GSM, GPRS or Bluetooth protocol according to standards such as IEEE 802.11a, 802.11b, 802.11g, or 802.11n), a power unit, and a memory unit. The memory unit can include RAM, ROM, one or more hard drives, one or more flash drives or some other suitable data storage elements such as disk drives, etc.

The processing unit controls the operation of the remote processing device 108 or the remote cloud server 110 and can be any suitable processor, controller or digital signal processor that can provide sufficient processing power depending on the desired configuration, purposes and requirements of the system 100.

The display can be any suitable display that provides visual information. For instance, the display can be a cathode ray tube, or a flat-screen monitor and the like if the remote processing device 108 or remote cloud server 110 is a desktop computer. In other cases, the display can be a display suitable for a laptop, tablet or handheld device, such as an LCD-based display and the like.

System 100 can generally be used for quantifying an injury recovery state based on sensor readings received from a plurality of sensors worn while a user is performing one or more specified activities. In some cases, system 100 may also track additional data derived from the sensor readings. The sensor readings, metric values, quantified injury recovery state values and derived data may be monitored, stored, and analyzed for the user. Aspects of the monitoring, storage and analysis of sensor readings, metric values and other data may be performed by one or more of the input unit 102, and/or a remote processing device 108, and/or the cloud server 110. For example, a non-transitory storage memory of one or more of the input unit 102, and/or a remote processing device 108, and/or the cloud server 110 can store a plurality of baseline values that can be compared with metric values and used to calculate a quantified injury recovery state value.

A remote cloud server 110 may provide additional processing resources not available on the input unit 102 or the remote processing device 108. For example, some aspects of processing the sensor readings acquired by the sensors 106 may be delegated to the cloud server 110 to conserve power resources on the input unit 102 or remote processing device 108. In some cases, the cloud server 110, input unit 102 and remote processing device 108 may communicate in real-time to provide timely feedback to a user regarding the sensor readings, metric values, quantified injury recovery state values and other related data.

In the example system 100 illustrated in FIG. 1, a single input unit 102 is shown. However, system 100 may include multiple input units 102 associated with the same individual. For example, system 100 may include two separate input units 102, each input unit 102 associated with one of the individual's legs. Sensor data from an individual input unit 102 may be used to determine metric values for an individual's corresponding leg.

In some examples, system 100 may include a separate sensing unit 105 (and optionally a separate IMU 112) for each lower limb of an individual. This may allow the metric values to be determined separately for each of the individual's feet. This may also allow one of the individual's lower limbs to be used as a baseline comparator for the individual where the other lower limb is injured. That is, the metric values generated from the sensor data obtained from the uninjured limb may be used to determine the baseline values against which the metric values from the injured limb are compared.

Alternatively, a single sensing unit 105 may be used to acquire force sensor data for both feet of an individual. This may be the case where the sensing unit 105 is incorporated into fitness equipment such as an exercise mat or treadmill. In such cases, the force sensor data acquired by the sensing unit 105 may be associated with individual feet through further processing by electronics module 104 and/or processing device 108.

The IMU 112 can be associated with a single foot. Accordingly, separate IMUs 112 may be provided for both feet. IMU data acquired by the IMU 112 associated with each foot may be used to associate the force sensor data acquired by a single sensing unit 105 with the corresponding foot.

Referring now to FIG. 2, shown therein is an example of an insole 200 that includes a sensing unit 202. The insole 200 is an example of an input device 102 that may be used in the system 100 shown in FIG. 1. The insole 200 may be the footwear insert described in International Patent Application Publication No. WO 2021/092676 A1 (Everett et al.), the entirety of which is incorporated herein by reference.

The insole 200 includes a sensor unit 202 and an optional liner 204. The liner 204 can provide a protective surface between the sensor unit 202 and an individual's foot. The liner 204 may have a slightly larger profile as compared to the sensor unit 202. That is, the outer perimeter 203 of the sensor unit 202 may be inwardly spaced from the outer perimeter 205 of the liner 204 by an offset 208. The offset 208 may be substantially consistent throughout the perimeter of the sensor unit 202 such that the sensor unit 202 is completely covered by the liner 204.

Optionally, the sensor unit 202 can include an IMU (not shown). The sensor unit 202 can also include a connector 206. The connector 206 may provide a coupling interface between the plurality of sensors 106 (and the optional inertial measurement unit) and an electronics module (not shown) such as electronics module 104. The coupling interface can allow signals from the sensors 106 and/or IMU to be transmitted to the electronics module. In some cases, the coupling interface may also provide control or sampling signals from the electronics module to the sensors 106 and/or IMU.

The arrangement of sensors 106 in the sensor unit 202 is an example of a sparse sensor array that may be used to collect force sensor data. In alternative examples, various different types of force sensors, force sensor arrays, and arrangements of force sensors may be used. For example, sensor units containing a dense force sensor array (e.g. a Pedar® insole or Tekscan® system) may also be used.

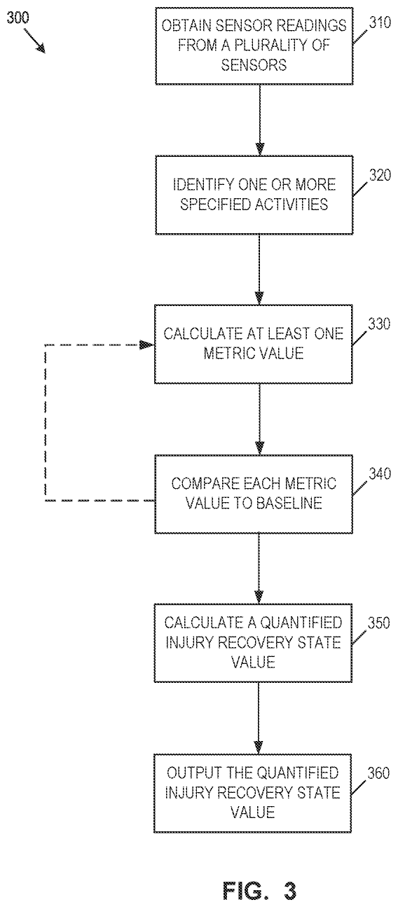

Referring now to FIG. 3, shown therein is an example method 300 for quantifying an injury recovery state for a user using sensor data from a plurality of sensors positioned to engage with the user. The method 300 may be used with a plurality of sensors configured to measure human movement or human activity, such as sensors 106 and IMU 112.

At 310, a plurality of sensor readings can be obtained from a corresponding plurality of sensors. At least some of the sensors can be positioned underfoot (i.e. underneath the foot) of a user performing a physical activity or movement. The plurality of sensors can be provided by one or more wearable devices. For example, the sensors can be provided using a wearable device such as an insole.

The sensors can include a plurality of force-sensing elements (force sensors) positioned underfoot. The force sensors can be configured to acquire force sensor data at locations underneath an individual's foot for one or both of the individual's lower limbs.

The force sensors can be positioned at specified locations on a carrier unit such as a wearable device or a piece of fitness equipment. The force sensors can be configured to measure force data relating to human activity. As shown in FIG. 2, the plurality of sensors may be force sensors provided at various locations of an insole. The force sensors can measure force applied to the insole while an individual performs various activities.

The plurality of sensors can also include one or more IMUs. Accordingly, the plurality of sensor readings acquired at 310 can include IMU sensor data received from the one or more IMUs.

Each inertial measurement unit (IMU) can be associated with the plurality of force sensors. For example, the IMU may be incorporated into the same wearable device as the plurality of force sensors. More generally, the IMU can be configured to collect IMU sensor data about a single lower limb of an individual. This IMU sensor data can be acquired for the same lower limb for which the sensor readings were obtained from the plurality of force sensors.

The sensor readings acquired at 310 may be acquired as a time-continuous set of sensor readings. This may provide a time-continuous set of sensor data that can be used to calculate metric values on a time-continuous basis (e.g. determining time-continuous force) and/or as discrete data values (e.g. a peak force value, an average jump height value). Depending on the nature of the sensors and the signal preprocessing performed, the time-continuous sensor data may be discretized, e.g. using an analog to digital conversion process. Even where the sensor data is discretized, the set of sensor data may allow the metric to be determined as (discretized) time-continuous values or discrete values.

The sensor readings can be associated with a specified activity (as described in further detail below at 320). That is, the sensor readings can be obtained while the individual is performing one or more specified activities.

Optionally, data collection can be initiated by the user directly or by a third-party overseeing data collection. For example, the sensor readings can be obtained in response to a user initiating a sensor collection session, e.g. through a mobile application on their mobile device. A user can initiate a data collection session while wearing the wearable device and prior to performing a specified activity. Sensor data can then be collected from the sensors in the wearable device while the user performs one or more activities.

Optionally, the plurality of sensor readings at 310 may be obtained after the user has incurred an injury. That is, the sensor readings may be used to assess post-injury performance by the user to evaluate the user's recovery from a lower limb injury.

Alternatively, the plurality of sensor readings may be obtained when a user has not yet incurred an injury. In this case, the sensor readings may be used to determine the user's potential for injury.

Optionally, a user may be authenticated prior to the collection of sensor data at 310. Since the sensor data can be obtained at various locations using wearable devices, it may not be possible for a clinician to observe data collection to ensure that the individual is personally performing the specified activities. To prevent individuals from falsifying data to obtain premature clearance to RTP (e.g. by having a non-injured friend perform the exercises), a user authentication process can verify the individual's identity prior to sensor data collection at 310.

At 320, one or more specified activities can be identified for the user. The specified activities can be identified as the one or more activities that the individual was performing while the sensor data was obtained at 310.

Optionally, each specified activity may be identified from a predefined list of potential activities. Table 1, described further below, illustrates an example list of potential activities.

The specified activities can be identified in various ways. An individual may manually identify a particular specified activity they are performing (e.g. walking) so that the sensor readings are associated with that specified activity set. For instance, a user may identify a specified activity by providing input to a mobile application while initiating sensor data collection. Optionally, the individual may select the specified activity from the predefined list of potential activities displayed through the mobile application.

Alternatively or in addition, each specified activity can be identified using the sensor data obtained at 310. For example, each specified activity may be automatically identified based on analysis of the sensor data.

Optionally, each specified activity may be automatically identified in real-time as sensor data is being collected.

An activity classification process may be applied to identify the specified activity associated with the sensor data or a subset of the sensor data. Activity-specific sensor data corresponding to an unidentified specified activity can be input to an activity classification model in order to identify that activity. The activity classification model can be configured to associate the activity-specific sensor data with a particular specified activity from a predefined list of potential activities.

The activity classification model can be trained using sensor data collected from a plurality of training users performing the potential activities. The activity classification model can be trained to identify which potential activity, among the list of potential activities is being performed using the sensor data. The activity classification model can be configured to identify the potential activity in real-time. Using an activity classification model to identify the specified activity in real-time may minimize the need for manual user input.

An example of an activity classification method that may be used to classify the sensor data is described in U.S. Pat. No. 11,526,749 entitled “METHOD AND SYSTEM FOR ACTIVITY CLASSIFICATION”, the entirety of which is incorporated herein by reference.

Method 300 can then proceed to step 330. As shown in FIG. 3, steps 330 and 340 may be repeated multiple times during the course of method 300. For example, steps 330 and 340 may be performed for each specified activity of the one or more specified activities identified at 320.

At 330, at least one metric value can be calculated for the user. Each metric value can be associated with a particular specified activity identified at 320.

In some cases, a plurality of metric values can be calculated for the user. The plurality of metric values can include one or more activity-specific metric values associated with each specified activity identified at 320.

Each metric value can be calculated based on activity-specific sensor data corresponding to a specified activity (identified at 320) from the plurality of sensor readings. Each metric value can correspond to a particular metric of interest associated with that specified activity.

Various different types of metric values can be calculated from the sensor readings acquired at 310. Some metric values can be calculated solely from the force sensor data, such as a rate of force development value, a peak force value, and an impulse value for example. Other metric values can be calculated solely from the IMU data. Other metric values can be calculated using both the force sensor data and IMU data. The metric values can be associated with the user and the activities performed by the individual while the sensor readings were collected.

The metric values can be defined as measurable biomechanical data that is related to (or appears to be related to) aspects of an injury recovery or potential injury for a user. Examples of metric values that can be calculated from the sensor readings include one or more of a center of pressure path value, a center of pressure velocity value, a rate of force development value, a peak force value, a jump height value, an impulse value, a rate of force dissipation value, a time to stabilization value, a distance value, or a ground contact time vs flight time value.

Depending on the type of metric values being calculated, the sensor data may be separated into a plurality of strides. This may be particularly relevant for metric values relating to running or walking movements.

Alternatively or in addition, the sensor data may be separated into a plurality of jumps. This may be particularly relevant for metric values relating to jumping movements.

The motion that an individual goes through while running or walking is typically referred to as a gait cycle. The gait cycle generally refers to the time period between the time when an individual's foot contacts the ground and the subsequent time when the same foot contacts the ground again. In some cases, the term gait cycle may also refer to the events that occur over that time period.

The term ‘stride’ is often used to refer to a single gait cycle for one foot. A stride can be divided into two phases: a stance phase and a swing phase. The stance phase generally refers to the period when the individual's foot remains, at least partially, in contact with a surface such as the ground. The swing phase generally refers to the period when the individual's foot is not in contact with the surface (e.g. as the foot swings in the air between periods when the foot is in contact with the ground).

The plurality of strides can be identified using data from the sensor readings. Each stride may be identified based on times when the individual's foot first contacts the ground (a foot-contact time) and/or times when the individual's foot leaves the ground (a foot-off time).

Each stride can be defined to correspond to a stride period. The stride period generally refers to the time period over which a single gait cycle extends. The endpoints of the stride period may vary in different implementations of method 300. For example, the stride period can be defined as the length of time between adjacent foot-contact times. Alternatively, the stride period can be defined as the length of time between adjacent foot-off times. The metric values may be determined for each stride based on the sensor readings acquired at 310 relating to the corresponding stride period. The metric values for a given stride may be determined as time-continuous stride values and/or overall stride values (e.g. mean values and/or peak values). Alternatively or in addition, metric values may be determined for multiple strides (e.g. mean values and/or peak values across multiple stride periods).

Force values and force-derived values may be determined based on aggregate force data for a corresponding stride or strides. The sensor readings may include corresponding force sensor values from each of the force sensors in the plurality of force sensors. The sensor readings may include a plurality of corresponding force sensor values from each of the force sensors in the plurality of force sensors at various time points throughout the time period of the stride or strides (e.g. as time-continuous sensor readings or sensor readings at discrete time steps). The aggregate force data may be determined based on the sensor values received from multiple force sensors in the plurality of force sensors over the time period of the stride or strides.

Various different methods can be applied to calculate the metric values based on the sensor readings acquired at 310. Example methods for determining parameters such as stride time, stride length, stride velocity, ground reaction forces, foot contact events, and so forth are described in further detail in Applicant's co-pending patent application Ser. No. 17/991,501 entitled “SYSTEM AND METHOD FOR ANALYZING FORCE SENSOR DATA” filed Nov. 21, 2022, the entirety of which is incorporated herein by reference.

The sensor readings obtained at 310 can include sensor data obtained from one or more data collection sessions for a given user. The metric values can be determined from the sensor readings from a single data collection session for a given user.

Alternatively, the metric values can be determined based on the sensor readings from a plurality of data collection sessions for a given user. For example, session-specific metric values can be determined for each data collection session based on the corresponding sensor readings. The metric values may be determined as average values of the session-specific metric values corresponding to the plurality of data collection sessions.

As noted above, various different metric values may be determined. In some cases, different metric values may be determined for different specified activities. For example, a rate of force development value may be determined for activities involving running or walking while a time to stabilization value may be determined for activities involving jumping. Table 1 shows an example list of specified activities and metrics of interest associated with each specified activity:

TABLE 1
Potential Specified Activities and
Corresponding Metrics of Interests

| Specified Activity | Metric of interest |
|---|---|
| Overhead squat | COP path |
| | COP velocity (AP and ML) |
| | Rate of force development (% diff) |
| | Peak force (% diff) |
| Single leg squat | COP path |
| | COP velocity (AP and ML) |
| | Rate of force development (% diff) |
| | Peak force (% diff) |
| Walk | COP path |
| | COP velocity (AP and ML) |
| | Rate of force development (% diff) |
| | Peak force (% diff) |
| Front leg weight bearing lunge | COP path |
| | COP velocity (AP and ML) |
| | Rate of force development (% diff) |
| | Peak force (% diff) |
| Countermovement jump | Jump height (m) |
| (single and double leg) | Rate of force development (% diff) |
| | Impulse (% diff) |
| | Peak force (% diff) |
| | COP path |
| | COP velocity (AP and ML) |
| Drop jump (single and double | Rate of force dissipation (% diff) |
| leg) | Time to stabilization (% diff) |
| | Peak force (% diff) |
| | Impulse (% diff) |
| | COP path |
| | COP velocity (AP and ML) |
| Single leg lateral hop | Distance (m) |
| | Time to stabilization (% diff) |
| | COP path |
| | COP velocity (AP and ML) |
| Single leg hops for distance | Distance (m) |
| | Ground contact time vs. flight time |
| | COP path |
| | COP velocity (AP and ML) |

As will be appreciated, the list of specified activities and associated metrics of interest shown in Table 1 is merely exemplary and other specified activities and/or metrics of interest may be used.

Optionally, metric values may only be calculated for activities that were performed correctly by an individual. Accordingly, the sensor data obtained at 310 can be evaluated to determine whether the individual was performing a specified activity correctly. Sensor data identified as corresponding to a specified activity that was performed incorrectly may be rejected (i.e. metric values would not be calculated from that sensor data).

The activity-specific sensor data corresponding to each specific activity can be identified. For each specific activity, the activity-specific sensor data can be evaluated to determine whether the specified activity was performed correctly or incorrectly. Metric values may only be calculated in response to determining that the specified activity was performed correctly.

Optionally, a specified activity may be identified as being performed correctly if the corresponding activity-specific sensor data is identified as corresponding to a potential specified activity. Evaluating the activity-specific sensor data and determining that the activity-specific sensor data corresponds to one of the potential activities (or corresponds above a specified confidence threshold) can indicate that the activity was performed correctly.

For example, where the activity-specific sensor data is provided as an input to an activity classification model and the model identifies the activity-specific sensor data as corresponding to a potential activity (optionally above a specified confidence threshold), this can indicate that the activity-specific sensor data corresponds to an activity that was performed correctly.

Optionally, the activity classification model can be further trained using training data for both “correctly performed” and “incorrectly performed” versions of each specified activity. The activity classification model may then be configured to identify activity-specific sensor data as corresponding to a correctly performed activity or an incorrectly performed activity.

Alternatively, an additional evaluation model can be developed to determine whether an activity is performed correctly or incorrectly. Once activity-specific sensor data has been associated with a particular activity, the evaluation model can be used to determine how closely the performance of the specified activity aligns with the proper technique using the activity-specific sensor data. For example, the evaluation model can be configured to score the activity performance based on the activity-specific sensor data on a scale from incorrectly performed to perfectly performed (e.g. a 0-5 scale, 0-100 scale or other scale). Activity-specific sensor data that falls below a specified performance level can then be identified as corresponding to an incorrectly performed activity and rejected.

Alternatively or in addition, conditions and/or restrictions can be defined for each specified activity. This may help exclude incorrect sensor data and/or sensor data relating to incorrectly performed activities. For example, following a drop jump, there may be a cut-off time (e.g. 8 seconds) to stabilization. If the time to stabilization following a drop jump is greater than 8 seconds, the activity-specified sensor data will be rejected. This may help reduce the processing required by rejecting sensor data that has little relevance to the user's injury recovery state. For instance, if a user performs a single leg drop jump and hops all around to try to regain their balance, the sensor data can be immediately rejected rather than processing the sensor data to calculate the user's time to stabilization. Similar conditions and/or restrictions can be defined for the other specified activities.

At 340, each metric value can be compared to a corresponding baseline metric value for the user. The comparison may provide insight into the user's current injury recovery state as compared to a baseline, non-injured state.

Optionally, the baseline metric values may be calculated from a different data collection period. Sensor data can be collected for the same user across multiple sensor data collection periods, including periods when the user is uninjured and injured. The sensor data from the various collection periods can be stored for later analysis. Accordingly, each baseline metric value can be calculated using baseline sensor readings obtained when the user is uninjured (e.g. prior to the user incurring an injury). This may allow metric values to be compared against baseline metric values calculated for the same limb.

Alternatively, baseline metric values may be calculated from the same data collection period. For example, a user may have an injury to one of their lower limbs (referred to as a first lower limb) while the other lower limb (referred to as a second lower limb) is uninjured. In such cases, the baseline metric value may be calculated using sensor data from the opposite non-injured limb (e.g. if the injured limb is the left ankle, the baseline metric value can be calculated using sensor data from the right ankle).

Each metric value can be calculated using first limb sensor readings in the plurality of sensor readings, where the first limb sensor readings were received from sensors positioned to monitor the first lower limb. Each baseline metric value can be calculated using second limb sensor readings in the plurality of sensor readings, where the second limb sensor readings are received from sensors positioned to monitor the second lower limb of the user.

At 350, a quantified injury recovery state value can be calculated based on the comparison of each metric value and the corresponding baseline metric value from 340. The quantified injury recovery state value can provide a quantitative indication of an individual's recovery from injury and/or potential for injury or reinjury.

Various different types of quantified injury recovery state values may be determined at 350 based on the comparison performed at 340. For example, the quantified injury recovery state value may be a current injury recovery state value. The current injury recovery state value can be determined using one or more baseline deviation values. Each baseline deviation value can be calculated from the comparison of a given metric value and the corresponding baseline metric value performed at 340. This can provide an indication of how close an individual is to a non-injured or recovered state.

A baseline deviation value may be determined as an absolute deviation value and/or a relative deviation value. For example, the baseline deviation value may be calculated as an absolute deviation value according to

diff_{metric}=|metric_{baseline}−metric_{current} (1)

where diff_{metric} represents the absolute deviation value, metric_{baseline} represents the baseline metric value, and metric_{current} represents a current metric value calculated at 330. Where the individual has an injury, the current metric value may also be referred to as a post-injury metric value.

A relative deviation value may be determined as a percentage deviation from the baseline metric value. For example, the baseline deviation value may be calculated as a relative deviation value according to

$$\% {diff}_{metric} = \vert \frac{{metric}_{baseline} - {metric}_{current}}{{metric}_{baseline}} \vert \times 100 \% \qquad ( 2 )$$

where % diff_{metric} represents the relative deviation value, metric_{baseline} represents the baseline metric value, and metric_{current} represents a current metric value calculated at 330. Once again, where the individual has an injury, the current metric value may also be referred to as a post-injury metric value.

The current injury recovery state can be determined using the baseline deviation values calculated for a user. For example, the current injury recovery state can be calculated as the absolute deviation value or relative deviation value for a particular metric of interest. Alternatively, the current injury recovery state can be determined using a combination of a plurality of baseline deviation values calculated for a user. For example, the current injury recovery state value can be determined using a weighted combination of the plurality of baseline deviation values. The weighted combination of the plurality of baseline deviation values can provide an absolute or relative current injury recovery state value based on the baseline deviation values for all metric of interests (for one specified activity or for all specified activities).

Certain metrics of interest may have a greater relevance to a particular specified activity. The baseline deviation values calculated for those metrics of interest when performing the particular specified activity may be weighted accordingly. For example, the current injury recovery state value can be determined using a weighted combination of the plurality of baseline deviation values according to:

% Wdiff_{metric}_{_{activity1}}=W₁(% diff_{metric1})W₂(% diff_{metric2})+ (3)

where % Wdiff_{metric}_{_{activity1}} represents the current injury recovery state value as a weighted percentage value that is a weighted combination of the baseline deviation values for a single specified activity, W₁ represents a first weight associated with a first relative deviation value (% diff_{metric1}) and W₂ represents a second weight associated with a second relative deviation value (% diff_{metric2}).

Certain specified activities can be identified as being more or less relevant to potential injury and/or injury recovery. Accordingly, the baseline deviation values (or weighted combination of the baseline deviation values) calculated for different specified activities may be weighted accordingly. For example, the current injury recovery state value can be determined using a weighted combination of the plurality of baseline deviation values from multiple specified activities according to:

% wdiff_{metric}_{_{allactivities}}=w₁(% Wdiff_{metric}_{_{activity1}})w₂(% Wdiff_{metric}_{_{activity2}})+ (4)

where % Wdiff_{metric}_{_{allactivities}} represents the current injury recovery state value as a weighted percentage value that is a weighted combination of the baseline deviation values for multiple specified activities, W₁ represents a first weight associated with a first specified activity, % Wdiff_{metric}_{_{activity1}} represents a weighted combination of the baseline deviation values for the first specified activity (which may be calculated using equation (3)), W₂ represents a second weight associated with a second specified activity, % Wdiff_{metric}_{_{activity2}} represents a weighted combination of the baseline deviation values for the second specified activity (which may also be calculated using equation (3)).

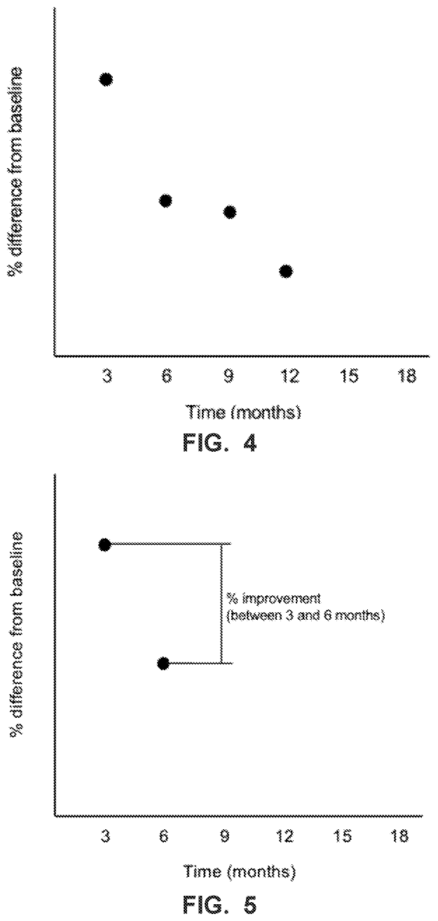

Optionally, sensor data may be collected from a user at various different points over time. Accordingly, metric values and quantified injury recovery state values can be calculated at different points for an individual. FIG. 4 shows an example plot of quantified injury recovery state values that may be generated for an individual over time. The plot shown in FIG. 4 illustrates a series of injury recovery state values calculated for an individual at different points in time post-injury (3 months, 6 months, 9 months and 12 months post-injury) showing the total deviation from baseline at each point in time.

The sensor data from previous recording sessions can be used to determine previous injury recovery state values for a user. This may allow the quantified injury recovery state value to reflect changes in the user's injury or potential for injury over time.

For each specified activity, at least one previous metric value can be determined for the user. Each previous metric value can be calculated using previous sensor readings previously obtained from the user while performing the specified activity. Each previous metric value can be compared to the corresponding baseline metric value for the user. A previous baseline deviation value can be calculated from the comparison of a given previous metric value and the corresponding baseline metric value. A previous injury recovery state value can then be determined using the one or more previous baseline deviation values. The previous injury recovery state value can be calculated in generally the same manner as the current injury recovery state value.

A user's current injury recovery state value and one or more previous injury recovery state values can be used to determine how the user's injury or potential for injury has changed over time. An injury improvement value can be determined by comparing the current injury recovery state value and the previous injury recovery state value. The injury improvement value may be calculated as the difference between the current injury recovery state value and the previous injury recovery state value according to

% improvement=|% diffₜ₂−% diffₜ₁| (5)

where % improvement represents the injury improvement value, % diffₜ₂ represents the current injury recovery state value, and % diffₜ₁ represents a previous injury recovery state value.

FIG. 5 shows an example plot of a current injury recovery state value (e.g. at the 6-month point) and a previous injury recovery state value (at the 3 month point) for an individual. The plot shown in FIG. 5 also illustrates an example injury improvement value indicating the level of improvement of the user's injury from 3 months post-injury to 6 months post-injury.

The current injury recovery state value and previous injury recovery state value can also be used to determine an injury improvement rate. The injury improvement rate can be determined as an estimated instantaneous rate of improvement in an individual's recovery from injury.

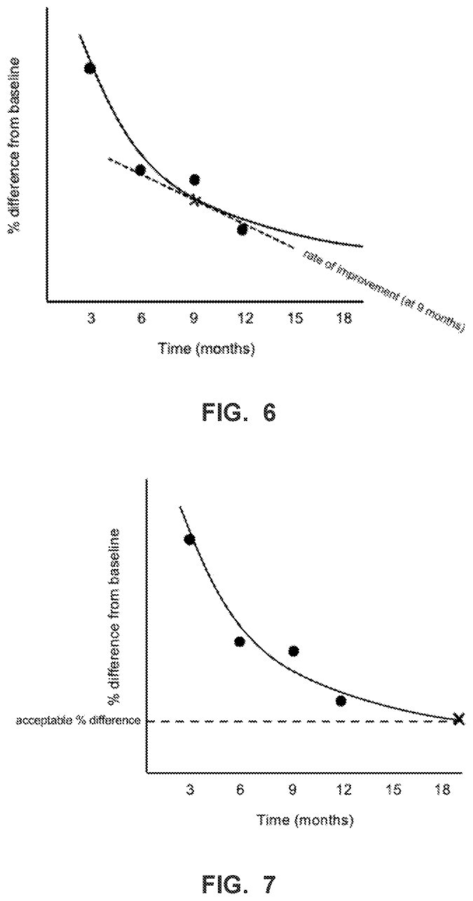

FIG. 6 shows an example plot of quantified injury recovery state values for an individual over time. As shown in FIG. 6, an injury improvement curve can be estimated based on the current and previous injury recovery state values generated for the individual. For example, the injury improvement curve can be determined by performing a regression on the current and previous injury recovery state values generated for the individual. An estimated instantaneous rate of improvement can be determined as the slope of the injury improvement curve at a given point in time (e.g. by taking a derivative of the equation of the line or using numerical methods to approximate the slope).

The current injury recovery state value and previous injury recovery state value can also be used to determine a predicted recovery time for the individual. The predicted recovery time can be determined as an estimate of the time until the user has recovered from a particular injury to a desired extent.

A potential recovery state value can be determined for the user using the at least one baseline metric value. The potential recovery state value can be defined to correspond directly to the at least one baseline metric value (i.e. representing a complete return to baseline for the individual). Alternatively, the potential recovery state value can be defined to include an acceptable deviation from the at least one baseline metric value. That is, an individual may be considered to be recovered even while the metric values calculated from the user still deviate from the corresponding baseline values by a predefined acceptable amount.

A predicted recovery time can be calculated using the current injury recovery state value and a previous injury recovery state value. The predicted recovery time can represent an estimate of how long it will take the user to return to an acceptable level from the current injury recovery state. The predicted recovery time may also be determined using an injury improvement curve.

FIG. 7 shows an example plot of quantified injury recovery state values for an individual over time including an injury improvement curve. As shown in FIG. 7, the estimated injury improvement curve intersects the potential recovery state value (i.e. acceptable % difference) just after 18 months post-injury. Accordingly, the predicted recovery time can be identified at that point.

Optionally, one or more metric values (from 330) can be compared to a metric value threshold. The metric value threshold may be defined as a desired minimum or maximum value of the corresponding metric that is expected to be associated with a recovered state. It can be determined whether the metric value satisfies the metric value threshold (e.g. is greater than a desired minimum value or less than a desired maximum value). A value threshold satisfaction output can be provided indicating whether the metric value satisfies the metric value threshold. This can provide an additional indication of whether an individual has recovered from an injury.

Optionally, multiple individual value threshold satisfaction outputs may be determined, e.g. a separate individual value threshold satisfaction output for each metric value determined at 330. Further optionally, a combined value threshold satisfaction output may be generated based on the individual value threshold satisfaction outputs. The combined value threshold satisfaction output can indicate whether every metric value satisfied the corresponding value threshold or if any failed to satisfy the corresponding value threshold.

Optionally, the quantified injury recovery state value can be used in conjunction with the determination of whether the metric value satisfies the metric value threshold in evaluating whether an individual has sufficiently recovered from an injury. For example, a recovered state may be identified based on a combination of the quantified injury recovery state value and one or more metric values satisfying corresponding metric value thresholds. A recovered state indication can be provided indicating whether the user appears to have reached a recovered state. The recovered state may be identified when the quantified injury recovery state value indicates that the user's metric values have returned to an acceptable level of deviation from the baseline values and each value threshold satisfaction output indicates that the corresponding metric value satisfies the metric value threshold.

As an example, the time to stabilization (TTS) metric can be considered. If the baseline deviation is minimal, for instance because both limbs have poor TTS (ex. >1s) but minimal difference, or if the user's pre-injury baseline had poor TTS, then the quantified injury recovery state value may suggest that the user is close to returning to their baseline, pre-injury state. However, the user's pre-injury baseline may have put the user at risk of the initial injury. Thus, attaining the same baseline value may still represent an increased risk of reinjury. Accordingly, the TTS value can be compared to a TTS threshold value (e.g. a maximum value of 1s). If the TTS value exceeds the maximum value, then the TTS metric value can be identified as failing to satisfy the TTS threshold value.

The sensor data obtained at 310 can also include sensor readings from an individual in both a fatigued state (e.g. after having performed a number of activities) and a non-fatigued state (e.g. when the individual is well-rested).

For example, the plurality of sensor readings obtained at 310 can include a first set of sensor readings corresponding to the user performing the one or more specified activities in a non-fatigued state and a second set of sensor readings corresponding to the user performing the one or more specified activities in a fatigued state. At least one non-fatigued metric value can be calculated for the user based on activity-specific sensor data corresponding to that specified activity from the first set of sensor readings. At least one fatigued metric value can be calculated for the user based on activity-specific sensor data corresponding to that specified activity from the second set of sensor readings. The quantified injury recovery state value can be calculated using the at least one fatigued metric value.

Metric values calculated for the fatigued and non-fatigued state can be used to generate a quantified injury recovery state value representing the individual's injury recovery state or risk of injury/re-injury using the metric values for the fatigued state. This may more accurately represent an individual's risk of injury when participating in an activity or event where they are likely to become fatigued while still participating.

At 360, the quantified injury recovery state value can be output. The quantified injury recovery state value may be provided to the user or to other individuals who may use the quantified injury recovery state value to assist the individual. Alternatively or in addition, the quantified injury recovery state value can be output to a non-transitory storage memory for later review, comparison, analysis or monitoring (e.g. as a previous injury recovery state value in a subsequent analysis).

The quantified injury recovery state value can be output directly through an output device to provide an individual (or clinician) with feedback on their performance and optionally an injury being monitored. For example, the quantified injury recovery state value may be transmitted to a mobile application on the individual's mobile device (e.g. a processing device 108). Providing a user with real-time feedback, or near-real-time feedback may assist the user in determining whether to proceed with further activities. For example, providing a user with such feedback can allow the individual to stop an activity when their injury recovery state indicates an increased risk of injury or re-injury.

Optionally, additional data relating to the quantified injury recovery state value can also be output. For example, some or all of the sensor readings, metric values, quantified injury recovery state values and related or derived data can be output. The additional data may be provided to the user or to other individuals who may use the additional data to assist the individual. Alternatively or in addition, the additional data can be output to a non-transitory storage memory for later review, comparison, analysis or monitoring.

For example, the sensor readings may be output and correlated with video data relating to the user's performance of one or more specific activities. The sensor readings (e.g. pressure data and/or IMU data) can be synchronized with the associated video data to assist in understanding how the user performs an activity. This may also help identify changes in the user's technique and how the changes may impact the user's injury recovery, which may provide a useful tool to help a user correct their technique to improve their injury recovery state.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for quantifying an injury recovery state for a user, the method comprising:
obtaining a plurality of sensor readings from a corresponding plurality of sensors provided in a wearable device, and while the wearable device is worn by the user while performing one or more specified activities;
identifying the one or more specified activities;
for each specified activity of the one or more specified activities,
calculating at least one metric value for the user based on activity-specific sensor data corresponding to that specified activity from the plurality of sensor readings, wherein each metric value corresponds to a particular metric of interest associated with that specified activity; and
comparing each metric value to a corresponding baseline metric value for the user; and
calculating a quantified injury recovery state value based on the comparison of each metric value and the corresponding baseline metric value.

2. The method of claim 1, wherein the plurality of sensor readings are obtained after the user has incurred an injury.

3. The method of claim 2, wherein each baseline metric value is calculated using baseline sensor readings obtained prior to the user incurring the injury.

4. The method of claim 2, wherein:
the injury is to a first lower limb of the user;
each metric value is calculated using first limb sensor readings in the plurality of sensor readings, the first limb sensor readings received from sensors positioned to monitor the first lower limb; and
each baseline metric value is calculated using second limb sensor readings in the plurality of sensor readings, the second limb sensor readings received from sensors positioned to monitor a second lower limb of the user, wherein the second lower limb is uninjured.

5. The method of claim 1, wherein the quantified injury recovery state value is a current injury recovery state value determined using at least one baseline deviation value, each baseline deviation value calculated from the comparison of a given metric value and the corresponding baseline metric value.

6. The method of claim 5, further comprising:
for each specified activity, determining at least one previous metric value for the user, each previous metric value calculated using previous sensor readings previously obtained from the user while performing the one or more specified activities;
comparing each previous metric value to the corresponding baseline metric value for the user; and
determining a previous injury recovery state value using at least one previous baseline deviation value, each previous baseline deviation value calculated from the comparison of a given previous metric value and the corresponding baseline metric value.

7. The method of claim 6, further comprising determining an injury improvement value or an injury improvement rate using the current injury recovery state value and the previous injury recovery state value.

8. The method of claim 6 further comprising:
identifying a potential recovery state value for the user using the at least one baseline metric value; and
calculating a predicted recovery time using the current injury recovery state value and the previous injury recovery state value.

9. The method of claim 1, further comprising:
identifying the activity-specific sensor data corresponding to each specific activity in the sensor readings;
for each specific activity,
determining whether the specified activity was performed correctly or incorrectly based on the activity-specific sensor data; and
only calculating the at least one metric value in response to determining that the specified activity was performed correctly.

10. The method of claim 1, wherein each specified activity is identified from a predefined list of potential activities and the method further comprises, for each specified activity:
evaluating the sensor readings to identify activity-specific sensor data corresponding to one of the potential activities;
identifying the one of the potential activities as that specified activity; and
determining that the specified activity was performed correctly in response to determining that the activity-specific sensor data corresponds to the one of the potential activities.

11. A system for quantifying an injury recovery state for a user comprising:
a plurality of sensors mountable to a user, wherein the plurality of sensors is configured to obtain a plurality of sensor readings from the user while the user is performing one or more specified activities;
a memory configured to store the plurality of sensor readings; and
one or more processors configured to:
identify the one or more specified activities;
for each specified activity of the one or more specified activities,
calculate at least one metric value for the user based on activity-specific sensor data corresponding to that specified activity from the plurality of sensor readings, wherein each metric value corresponds to a particular metric of interest associated with that specified activity; and
compare each metric value to a corresponding baseline metric value for the user; and
calculate a quantified injury recovery state value based on the comparison of each metric value and the corresponding baseline metric value.

12. The system of claim 11, wherein the plurality of sensor readings are obtained after the user has incurred an injury.

13. The system of claim 12, wherein the one or more processors is configured to calculate each baseline metric value using baseline sensor readings obtained prior to the user incurring the injury.

14. The system of claim 12, wherein the injury is to a first lower limb of the user and the one or more processors is configured to:
calculate each metric value using first limb sensor readings in the plurality of sensor readings, the first limb sensor readings received from sensors positioned to monitor the first lower limb; and
calculate each baseline metric value using second limb sensor readings in the plurality of sensor readings, the second limb sensor readings received from sensors positioned to monitor a second lower limb of the user, wherein the second lower limb is uninjured.

15. The system of claim 11, wherein the one or more processors is configured to determine the quantified injury recovery state value as a current injury recovery state value using at least one baseline deviation value, wherein each baseline deviation value is calculated from the comparison of a given metric value and the corresponding baseline metric value.

16. The system of claim 15, wherein the one or more processors is configured to:
for each specified activity, determine at least one previous metric value for the user, each previous metric value calculated using previous sensor readings previously obtained from the user while performing the one or more specified activities;
compare each previous metric value to the corresponding baseline metric value for the user; and
determine a previous injury recovery state value using at least one previous baseline deviation value, each previous baseline deviation value calculated from the comparison of a given previous metric value and the corresponding baseline metric value.

17. The system of claim 16, wherein the one or more processors is configured to determine an injury improvement value or an injury improvement rate using the current injury recovery state value and the previous injury recovery state value.

18. The system of claim 16 wherein the one or more processors is configured to:
identify a potential recovery state value for the user using the at least one baseline metric value; and
calculate a predicted recovery time using the current injury recovery state value and the previous injury recovery state value.

19. The system of claim 11, wherein the plurality of sensors comprises a force-sensing element and/or an inertial measurement unit.

20. The system of claim 11, wherein the plurality of sensors is disposed on a wearable device, and wherein the wearable device is an insole.
